(19) United States
(12) Patent Application Publication
(10) Pub. No.: US 2026/0365594 A1
(43) Pub. Date: Nov. 19, 2026

(54) MANUFACTURE OF AN ETHYLENE-DERIVED CHEMICAL OF INTEREST IN COMBINATION WITH PRODUCTION OF THERMAL ENERGY

(71) Applicant: BASF SE, Ludwigshafen am Rhein (DE)

(72) Inventors: Stefan WILLERSINN, Houston, TX (US); Christian WEINEL, Ludwigshafen am Rhein (DE); Daniel KECK, Ludwigshafen am Rhein (DE); Dieter RAHN, Ludwigshafen am Rhein (DE); Johannes Lazaros Friedrich ELLER, Ludwigshafen am Rhein (DE)

(21) Appl. No.: 19/141,087

(22) PCT Filed: Dec. 18, 2023
(86) PCT No.: PCT/EP2023/086355

§ 371 Date: Jun. 19, 2025

(30) Foreign Application Priority Data
Dec. 20, 2022 (EP) 22215084.9

(51) Int. Cl. C07C 1/24; C07C 2/06; C07C 5/27; C07C 6/04; C07C 7/04; C07C 29/48; C07C 29/56; C07C 45/50

(52) U.S. Cl. CPC C07C 1/24; C07C 2/06; C07C 5/2767; C07C 6/04; C07C 7/04; C07C 29/48; C07C 29/56; C07C 45/50

(57) ABSTRACT

A process for the manufacture of a chemical of interest comprises subjecting a feedstock comprising a renewably-sourced ethanol to dehydration to produce a renewably-sourced ethylene stream, which is subjected to an olefin-interconversion to obtain one or more renewably-sourced C₃₋₄-olefins, selected from propylene, n-butenes and isobutene. Further, the renewably-sourced C₃₋₄-olefin is subjected to a chemical conversion or sequence of chemical conversions to obtain the chemical of interest, the chemical conversion or sequence of chemical conversions producing one or more crude product streams and, optionally, one or more crude intermediate product streams. Subsequently, at least one of the crude product stream(s) and optional intermediate crude product stream(s) is subjected to purification producing one or more purified product streams, one or more waste streams and, optionally, one or more purified intermediate product streams. Then, at least one of the waste streams is incinerated to produce thermal energy. The process provides a reaction scheme that provides renewably-sourced ethylene-derived chemical of interest, while minimizing both fossil carbon dioxide emissions and consumption of renewably-sourced feedstock.

The present invention relates to a process for the manufacture of an ethylene-derived chemical of interest in combination with production of thermal energy.

The manufacture of many chemical derivatives is typically performed with a multi-step chemical reaction process which produces a crude product stream containing not only the desired chemical derivative, but also other compounds including, but not limited to, unreacted raw materials, intermediate reaction products, by-products and impurities. Thus, one of the final or intermediate steps in the multi-step process for manufacture of a chemical derivative involves purification, such as by distillation, of the crude product stream to separate the desired product from the majority of the other compounds.

Where the other compounds do not warrant further separation or recycling to one or more steps of the multi-step process the waste streams have either been discarded or incinerated and thus exploited for their calorific value. Since currently the carbonaceous raw materials used in the manufacture of most chemical derivatives are almost exclusively of fossil origin such as natural gas or crude oil, the incineration of waste streams is a source of CO₂ emissions that contribute to the carbon footprint of a chemical production site. The situation is aggravated with chemical reactions that inherently have a high rate of by product formation since the yield loss caused by the by products is accompanied by a high rate of climate-damaging emissions when the by products are burned.

There are increasing regulatory pressures to reduce emissions of carbon dioxide, especially fossil carbon dioxide. For example, a producer may be given the choice of physically reducing their carbon dioxide emissions, or paying a carbon tax in a carbon tax system or some combination of each. By utilizing biogenic carbon for fuel, CO₂ emissions associated with the combustion do not contribute to taxable carbon emissions because carbon is recycled to grow more biomass.

Ethylene is a cornerstone of the modern petrochemical industries. Important ethylene derivatives (at the end of their respective chains) include (meth)acrylic acid, (meth)acrylic esters, isononanols, ethylhexanol, and ethylene glycols. One of the problems faced by the manufacture of chemicals and intermediates from ethylene is that the starting raw materials are from fossil fuels, such as natural gas or crude oil, which are non-renewable feedstocks. Steam cracking, which employs petroleum fractions and natural gas liquids as feedstocks, is the dominant method for large-scale ethylene production worldwide.

Lower olefins, such as isobutylene or propylene, are of significant interest for industrial and chemical applications. Isobutylene, also known as isobutene or 2-methylpropene, is a hydrocarbon of significant interest that is widely used as an intermediate in the production of industrially important products, including para-xylene, jet fuel blendstocks, gasoline oxygenates, isooctane, methacrolein, methyl methacrylate, and butyl rubber. Propylene is a hydrocarbon of significant interest that is widely used as an intermediate in the production of acrylic acid. Historically, lower olefins have been obtained through the catalytic or steam cracking of fossil fuel feedstocks.

The production of ethylene and ethylene derivatives compounds would benefit from the replacement of at least a part of the carbonaceous raw materials of fossil origin by renewable resources, such as carbonaceous matter derived from biomass. Of particular interest is the ethanol feedstock which is produced from renewable resources. Such renewably-sourced ethanol, also referred to as “bioethanol” or “hydrous fuel alcohol” can be prepared in large quantities from organic waste or biomass via fermentation. The different feedstocks for producing ethanol may be sucrose-containing feedstocks, e.g., sugarcane, starchy materials, e.g., corn, starch, wheat, cassava, lignocellulosic biomass, e.g., switchgrass, and/or agricultural waste. The purification or isolation of bioethanol is frequently carried out by complicated, multistage distillation.

The invention seeks to advise a reaction scheme that provides renewably-sourced ethylene-derived chemicals of interest, while minimizing both fossil carbon dioxide emissions and consumption of renewably-sourced feedstock.

The invention relates to a process for the manufacture of a chemical of interest, said process comprising the steps of:
a) subjecting a feedstock comprising a renewably-sourced ethanol to dehydration to produce a renewably-sourced ethylene stream;
b) subjecting the renewably-sourced ethylene stream to an olefin-interconversion, to obtain one or more renewably-sourced C₃₋₄-olefins, selected from propylene, n-butenes and isobutene; the olefin-interconversion comprising (i) and, where required, one or both of (ii) and (iii):
   (i) ethylene dimerization to obtain n-butenes;
   (ii) metathesis reaction between n-butenes obtained according to (i) and ethylene to obtain propylene; and
   (iii) isomerization of n-butenes obtained according to (i) to obtain isobutene; and
c) subjecting the renewably-sourced C₃₋₄-olefin to a chemical conversion or sequence of chemical conversions to obtain the chemical of interest, the chemical conversion or sequence of chemical conversions producing one or more crude product streams and, optionally, one or more crude intermediate product streams,
d) subjecting at least one of the crude product stream(s) and optional intermediate crude product stream(s) to purification producing one or more purified product streams, one or more waste streams and, optionally, one or more purified intermediate product streams,
e) incinerating at least one of the waste streams to produce thermal energy.

At least one purified product stream constitutes the chemical of interest.

It should be appreciated that full or partial replacement of fossil ethylene and C₃₋₄-olefins by their renewably-sourced counterparts at the beginning of the processing chain reduces the emission of fossil-based carbon dioxide resulting from the combustion of downstream side-products. If a renewably-sourced olefin is employed as the starting material of a reaction sequence leading to a chemical of interest, the incineration of associated waste streams with generation of CO₂ can be regarded as a combustion of the renewably-sourced ethanol which is shifted to the incineration step. The key feature of this preferred embodiments is the specific combination of the renewably-sourced olefin production with the incineration of unavoidable waste streams. The thermal energy thus generated can be regarded as “green energy” because the heat used for its generation can be attributed to the combustion of renewably-sourced ethanol, thus no fossil resources are required.

Of course, one could also directly combust a certain amount of renewably-sourced ethanol (i.e. using it as a bio fuel) to generate the required heat. However, once combusted, the renewably-sourced ethanol could not be used anymore as a starting material for chemical synthesis. This amount of renewably-sourced ethanol can be saved by the specific combination according to this preferred embodiment. Put otherwise, the combustion of part of the renewably-sourced ethanol to generate heat is shifted to a later production step, namely the incineration of waste streams, as part of the inevitable yield losses there. Thus, this embodiment incorporates the use of renewably-sourced ethanol as a starting material for chemical synthesis and as a bio fuel at the same time.

In preferred embodiments, the ethanol to olefin production steps a) and b) are combined with those downstream chemical conversions that have limited selectivity or are non-selective with respect to the chemical of interest or, in other words, are accompanied by a substantial number of undesirable reactions. Such chemical conversions have a considerable low yield of the desired chemical of interest and upon purification of the crude product stream a substantial amount of waste stream is obtained in addition to the purified product stream.

A chemical conversion with limited selectivity or non-selective chemical conversion is deemed a conversion having a selectivity of less than 95%, preferably 90% or less. Selectivity is defined as the percentage of the renewably-sourced C₃₋₄-olefin (or mixed renewably-sourced/fossil-sourced C₃₋₄-olefin) converted which becomes the desired chemical of interest. The overall selectivity of a sequence of chemical conversions to obtain the chemical of interest may be calculated by multiplying the partial selectivities for each of the single-step conversions. Preferably, the selectivity is in the range of 60% to 90% or 70% to 85%. The lower limit of such ranges constitutes a compromise of obtaining a significant amount of waste for incineration but at the same time still having a significant yield of the chemical of interest.

It is particularly preferred when the waste that is generated in the production of the respective chemical of interest and being subjected to incineration according to step e) has a high lower heating value (“LHV”). The lower heating value is the gross heating value minus the latent heat of vaporization of the water vapor formed by the combustion of components in the waste that include the element hydrogen. The lower heating value is the thermal energy that is produced by the combustion of 1 kg of waste considering energy losses being required to vaporize the water. Such energy losses are not released as heat. The LHV is measured in a bomb calorimeter. The combustion of a stoichiometric mixture of a specific amount of waste and oxygen in a steel container at 25 °C is initiated by an ignition device and the reactions allowed to complete. After completion of the reaction, the steel container, including the combusted waste, is cooled to 150 °C. The LHV is thus defined as the amount of heat released by combusting a specific amount of waste (initially at 25 °C) and returning the temperature of the combustion products to 150 °C.

Preferably the waste stream that is incinerated according to step e) has a lower heating value LHW in the range of from 10 to 45 MJ, preferably 15 to 45 MJ, more preferably 20 to 40 MJ per kg of waste.

In preferred embodiments, the percentage of biogenic carbon in the waste that is incinerated according to step e) is in the range from 20 to 100, preferably 40 to 90, more preferably 50 to 85 wt.-% based on the total carbon (biogenic and fossil carbon) in the waste.

Biogenic carbon may be distinguishable from fossil carbon on the basis of carbon-isotopic fingerprinting and/or ¹⁴C (fM). There are three naturally occurring isotopes of carbon: ¹²C, ¹³C, and ¹⁴C. These isotopes occur in above-ground total carbon at fractions of 0.989, 0.011, and ^{10~12}, respectively. The isotopes ¹²C and ¹³C are stable, while ¹⁴C decays naturally to ¹⁴N with a half-life of 5730 years. The isotope ¹⁴C originates in the atmosphere, due primarily to neutron bombardment of ¹⁴N caused ultimately by cosmic radiation. Because of its relatively short half-life (in geologic terms), ¹⁴C occurs at extremely low levels in fossil carbon.

Alternatively, the ratio of fossil carbon to biogenic carbon may, of course, be assessed on the basis of the biogenic/fossil carbon ratio of the streams entering the reaction in which the waste stream is generated.

Preferably, the process comprises f) transferring at least part of the thermal energy to a water stream to generate a heated steam stream. Conveniently, the heated steam stream is used to provide heat to one or more heat-accepting processes. Modern industrial chemical production sites are integrated with multiple processes and plants to produce various chemical products. A “heat accepting process” can be any process in a respective industrial chemical production site that requires steam. The steam can be utilized in equipment such as heat exchangers, steam turbines, reboilers, and the like. This can be for instance any or all of steps a), b)-(i), b)-(ii) and b)-(iii) but is not limited thereto. A “distributed steam grid” is used for heat transfer between different processes within a site and thus the integration and effective use of heat. The thermal energy can generate steam by vaporization of water, preferably in the form of steam condensate. The steam is fed into the steam grid. The thermal energy can additionally be utilized in order to raise steam to a high pressure level, of 4 bar gauge or higher, as is typically provided in the steam lines in industrial plants.

In an embodiment, the heated steam stream is used to provide at least a portion of the heat consumed in at least one of steps a) and b), more specifically in at least one of steps a), b)-(i), b)-(ii) and b)-(iii). Thereby, the economic feasibility of ethanol dehydration and olefin conversion steps can be improved because less fossil energy is required for steam generation.

Purification to obtain one or more purified product streams or one or more purified intermediate product streams may involve distillation of a crude product stream(s) or crude intermediate product stream(s). The various species encountered in a chemical reaction or reaction sequence may undergo a host of side reactions, which generate color forming species, oligomers, and various decomposition products or the like. These are generally removed during work-up, e.g., by distillation, yielding light boiler and/or high boiler fractions in addition to the desired product. The light boiler or high boiler fractions are used for their calorific value, i.e. combusted as fuel. Due to their generally higher calorific value, high boilers or distillation bottoms streams are preferred waste streams that are directed to incineration.

In an embodiment of the process according to the invention, the renewably-sourced C₃₋₄-olefin is propylene, and step c) comprises hydroformylation of the propylene to produce a crude aldehyde stream comprising n-butyraldehyde and isobutyraldehyde; and step d) comprises purification of the crude aldehyde stream to produce a purified aldehyde stream and a waste stream.

In another embodiment of the process according to the invention, the renewably-sourced C₃₋₄-olefin is isobutene, and step c) comprises (c-i) through (c-v) and step d comprises (d-i) through (d-iii):
(c-i) a reaction of isobutene with formaldehyde to produce a crude isoprenol stream, (d-i) purification of the crude isoprenol stream to produce a purified isoprenol stream and a first waste stream;
(c-ii) an oxidation reaction of the purified isoprenal stream to produce a crude isoprenal stream, (d-ii) purification of the crude isoprenal stream to produce a purified isoprenal stream and a second waste stream;
(c-iii) isomerization of the purified isoprenol stream to produce prenol,
(c-iv) isomerization of the purified isoprenal stream to produce prenal,
(c-v) reaction of the prenol with prenal to produce a crude citral stream, (d-iii) purification of the crude citral stream to produce a purified citral stream and a third waste stream. The first waste stream, second waste stream and third waste stream may be combined and directed to incineration.

The expressions “renewable” or “renewably-sourced” in relation to a chemical compound are used synonymously and mean a chemical compound comprising a quantity of renewable carbon, i.e., having a reduced or no carbon content of fossil origin. Renewable carbon entails all carbon sources that avoid or substitute the use of any additional fossil carbon from the geosphere. Renewable carbon can come from the biosphere, atmosphere or technosphere—but not from the geosphere. Thus, the expresssion “renewable” or “renewably-sourced” includes, in particular, biomass-derived chemical compounds. It also includes compounds derived from waste such as polymer residues, or from waste streams of chemical production processes.

The expression “chemical of interest” collectively refers to any desired compound appearing in a value chain starting out from ethylene.

Bioethanol is a preferred form of renewably-sourced ethanol, although the scope of the invention is not limited to the use of bioethanol.

In the present invention, bioethanol refers to the ethanol obtained from a biomass feedstock, such as plant or non-crop feedstock containing a carbon source that is convertible to ethanol, for example by microbial metabolism. Typical carbon source examples are starch, sugars like pentoses or hexoses, such as glucose, fructose, sucrose, xylose, arabinose, or degradation products of plants, hydrolysis products of cellulose or juice of sugar canes, beet and the like containing large amounts of the above components.

Biomass feedstock can originate from several sources. Bioethanol production may be based on food crop feedstocks such as corn and sugar cane, sugarcane bagasse, cassava (first generation biofeedstock).

Another source of biomass feedstock is lignocellulosic materials from agricultural crops (second-generation biofeedstock). Potential feedstocks include agricultural residue by-products such as rice, straw (such as wheat, oat and barley straw), rice husk, and corn stover. Biomass feedstock may also be waste material from the forest products industry (wood waste) and saw dust or produced on purpose as an ethanol crop. Switchgrass and napier grass may be used as on-purpose crops for conversion to ethanol.

The first-generation bioethanol is produced in four basic steps:
(1) Enzymatic saccharification or hydrolysis of starch into sugars
(2) Microbial fermentation of sugars
(3) Purification by distillation to give hydrous ethanol
(4) Dehydration (water removal) to produce anhydrous ethanol

Second-generation feedstocks are considered as renewable and sustainable carbon source. Pretreatment of this feedstock is an essential prerequisite before it is subjected to enzymatic hydrolysis, fermentation, distillation, and dehydration. Pretreatment involves milling and exposure to acid and heat to reduce the size of the plant fibers and hydrolyze a portion of the material to yield fermentable sugars. Saccharification utilizes enzymes to hydrolyze another portion to sugar. Finally, fermentation by bioengineered microorganisms converts the various sugars (pentoses and hexoses) to ethanol. The production of bioethanol is well-known and carried out on an industrial large scale.

Renewably-sourced ethanol can also be obtained from carbon-containing waste materials like waste products from the chemical industry, garbage and sewage sludge. The production of ethanol from waste materials can be done by gasification to syngas and catalytic conversion thereof the ethanol, see for example Recent Advances in Thermo-Chemical Conversion of Biomass, 2015, Pages 213-250, https://doi.org/10.1016/B978-0-444-63289-0.00008-9, and Nat Commun 11, 827 (2020), https://doi.org/10.1038/s41467-020-14672-8.

### Dehydration of Renewably-Sourced Ethanol

As a first step, the invention involves the dehydration of renewably-sourced ethanol. The production of ethylene by catalytic dehydration of ethanol is a well-known process. The reaction is commonly carried out at 300 to 400 °C and moderate pressure in the presence of a catalyst. Catalytic effects are reviewed in Ind & Eng Chem Research, 52, 28, 9505-9514 (2013), Materials 6, 101-115 (2013) and ACS Omega, 2, 4287-4296 (2017). Examples for catalysts are activated alumina or silica, phosphoric acid impregnated on coke, heteropoly acids (HPA salts), silica-alumina, molecular sieves such as zeoliths of the ZSM-5 type or SAPO-11 type, other zeolites or modified zeolites of various molecular structures with zeoliths and HPA salts being preferred.

Ethanol dehydration is, for example described in WO 2009/098268, WO 2010/066830, WO 2009/070858 and the prior art discussed therein, WO 2011/085223 and the prior art discussed therein, U.S. Pat Nos. 4,234,752, 4,396,789, 4,529,827 and WO 2004/078336.

The ethanol dehydration reaction is in general carried out in the vapor phase in contact with a heterogeneous catalyst bed using either fixed bed or fluidized bed reactors. For fixed bed reactors, the operation can be either isothermal (with external heating system) or adiabatic (in the presence of a heat carrying fluid). The feedstock is vaporized and heated to the desired reaction temperature; the temperature drops as the reaction proceeds in the reactor. Multiple reactor beds are usually used in series to maintain the temperature drop in each bed to a manageable range. The cooled effluent from each bed is further heated to bring it to the desired inlet temperature of the subsequent beds. Moreover, a portion of the water is recirculated along with fresh and unreacted ethanol. The presence of water helps in moderating the temperature decrease in each bed.

Prior to dehydration, the renewably-sourced ethanol feedstock may be sent to a pretreatment section to remove mineral contaminants, which would otherwise be detrimental to the downstream catalytic reaction. The pretreatment may involve contacting the renewably-sourced ethanol feedstock with cation and/or anion exchange resins. After a certain period of operation, the resins may be regenerated by passing a regenerant solution through the resin bed(s) to restore their ion exchange capacity. Two sets of beds are preferably operated in parallel to maintain continuous operation. One set of resin beds is suitably regenerated while the other set is being used for pretreatment.

In the isothermal design, the catalyst is placed inside the tubes of multitubular fixed-bed reactors which arranged vertically and surrounded by a shell (tube and shell design). A heat transfer medium, such as molten salts or oil, is circulated inside the shell to provide the required heat. Baffles may be provided on the shell side to facilitate heat transfer. The cooled heating medium is heated externally and is recirculated. The temperature drop on the process side can be reduced as compared to the adiabatic reactor. A better control on the temperature results in increased selectivity for the ethylene formation and reduction in the amount of undesirable by-products. The temperature is maintained at approximately constant levels within the range of 300° to 350 °C. Ethanol conversion is typically between 98 and 99%. The selectivity to ethylene is preferably greater than 90 mol-%, more preferably greater than 93 mol-%, most preferably greater than 95 mol-%, such as 95 to 99 mol-%. Because of the rate of coke deposition, the catalyst must be regenerated frequently. Depending on the type of catalyst used, the cycle life is between 3 weeks and 4 months, followed by regeneration, for example for 3 days.

In the adiabatic design, the endothermic heat of reaction is supplied by a preheated inert diluent such as steam. Three fixed-bed reactors may typically be used, with intermediate furnaces to reheat the ethanol/steam mixed feed stream to each reactor. Feeding steam with ethanol results in less coke formation, longer catalyst activity, and higher yields.

A further process is a fluidized-bed process. The fluidized-bed system offers excellent temperature control in the reactor, thereby minimizing by-product formation. The heat distribution rate of the fluidized bed operation approaches isothermal conditions. The endothermic heat of reaction is supplied by the hot recycled silica-alumina catalyst returning from the catalyst regenerator. Thus, external heating of the reactor is not necessary.

After dehydration, the reaction mixture is subjected to a separation step. The general separation scheme consists of quickly cooling the reaction gas, for example in a water quench tower, which separates most of the by-product water and the unreacted ethanol from ethylene and other light components which, for example exit from the top of the quench tower. In one type of separation scheme, the water-washed ethylene stream is immediately caustic-washed, for example in a column, to remove traces of CO₂. The gaseous stream may enter a compressor directly or pass to a surge gas holder first and then to a gas compressor. After compression, the gas is cooled with refrigeration and then passed through an adsorber with, for example activated carbon, to remove traces of heavy components, (e.g., C4s), if they are present. The adsorber is followed by a desiccant drying and dust filtering step before the ethylene product leaves the plant. This separation scheme produces 99%+ purity ethylene. If desired, the ethylene is further purified by caustic washing and desiccant-drying, and fractionated in a low-temperature column to obtain the final product.

Several commercial processes are currently in operation, developed by Braskem, Chematur, British Petroleum (BP), and Axens together with Total and IFPEN. The processes differ, e.g., in their process conditions, catalysts and adopted heat integration scheme. The process by BP (now Technip) is called Hummingbird. In this process, a heteropoly acid is used as catalyst, and the reactor operates at 160 to 270 °C and 1 to 45 bar. The unreacted ethanol in recirculated to the reactor. The process developed by Axens is called Atol. Two fixed bed adiabatic reactors, operating at 400 to 500 °C, are used. Chematur's process operates with four adiabatic tubular reactors. Syndol catalysts, with the main components of Al₂O₃-MgO/SiO₂, are employed in this process that was developed by American Halcon Scientific Design, Inc. in the 1980s. In the Braskem process, the adiabatic reactor feed is diluted with steam to a large extent. In such a process, the reactor operates at 180 to 600 °C , preferably 300 to 500 °C , and at 1.9 to 19.6 bar. An alumina or silica-alumina catalyst is used. The Braskem process is described in more detail in U.S. Pat. No. 4,232,179. A process control in accordance with the Braskem process is particularly preferred.

### Dimerization of Ethylene

The process of the invention involves an ethylene-dimerization to obtain n-butenes in accordance with step b)-(i) above. Any known method can be used for ethylene dimerization to produce n-butenes. A review on dimerization and oligomerization chemistry and technology is given in Catalysis Today, vol. 14(no. 1), Apr. 10, 1992.

Expediently, step b)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream; and
fractionating the effluent to recover a stream consisting essentially of n-butenes, a stream consisting essentially of heavier olefins, and an optional ethylene stream.

The dimerization catalyst may be homogeneous or heterogeneous. Typical dimerization catalysts are titanium or nickel compounds activated with alkyl aluminium compounds. In general, the Ti(IV) valency is stabilized by selecting the appropriate ligands, alkyl aluminium compound, the solvent polarity and the Al/Ti ratio. Nickel compounds that can catalyse the selective production of butenes are typically based on cationic nickel salts stabilised with phosphine and activated with alkyl aluminium compounds.

In one embodiment, the oligomerization of ethylene is implemented in the presence of a catalytic system in the liquid phase comprising a nickel compound and an aluminum compound. Such catalytic systems are described in the documents FR 2 443 877 and FR 2794 038. The Dimersol ET process is based on this technology and leads to the industrial production of olefins.

Thus, in one embodiment, the oligomerization of ethylene is implemented in the presence of a catalytic system comprising:
i) at least one bivalent nickel compound,
ii) at least one hydrocarbyl aluminum dihalide of formula AIRX₂, in which R is a hydrocarbyl radical comprising 1 to 12 carbon atoms, such as alkyl, aryl, aralkyl, alkaryl or cycloalkyl, X is a chlorine or bromine atom, and
iii) optionally a Brønsted organic acid.

As the bivalent nickel compound, nickel carboxylates of general formula (R¹COO)₂Ni are preferably used, where R¹ is an optionally substituted hydrocarbyl radical, for example alkyl, cycloalkyl, alkenyl, aryl, aralkyl, or alkaryl, containing up to 20 carbon atoms, preferably a hydrocarbyl radical of 5 to 20 carbon atoms, preferably 6 to 18 carbon atoms. Suitable bivalent nickel compounds include: chloride, bromide, carboxylates such as octoate, 2-ethylhexanoate, decanoate, oleate, salicylate, hydroxydecanoate, stearate, phenates, naphthenates, and acetyl acetonates. Nickel 2-ethylhexanoate is preferably used.

The hydrocarbyl aluminum dihalide compound corresponds to the formula AIRX₂, in which R is a hydrocarbyl radical comprising 1 to 12 carbon atoms, such as alkyl, aryl, aralkyl, alkaryl or cycloalkyl, and X is a chlorine or bromine atom. As examples of such compounds, it is possible to mention ethylaluminum sesquichloride, dichloroethyl aluminum, dichloroisobutyl aluminum, chlorodiethyl aluminum or mixtures thereof. According to a preferred method, a Brønsted organic acid is used. The Brønsted acid compound corresponds to the formula HY, where Y is an organic anion, for example carboxylic, sulfonic or phenolic. Halocarboxylic acids of formula R²COOH in which R² is a halogenated alkyl radical are preferred, in particular those that contain at least one alpha-halogen atom of the group —COOH with 2 to 10 carbon atoms in all. Preferably, a haloacetic acid of formula CXₚH₃₋ₚ—COOH is used, in which X is fluorine, chlorine, bromine or iodine, with p being an integer from 1 to 3. By way of example, it is possible to cite the trifluoroacetic, difluoroacetic, fluoroacetic, trichloroacetic, dichloroacetic, and chloroacetic acids. It is also possible to use arylsulfonic, alkylsulfonic, and fluoroalkylsulfonic acids, and picric acid and nitroacetic acid. Trifluoroacetic acid is preferably used.

The three components of the catalytic formula can be mixed in any order. However, it is preferable first to mix the nickel compound with the Brønsted organic acid, and then next to introduce the aluminum compound. The molar ratio of the hydrocarbyl aluminum dihalide to the nickel compound, expressed by the Al/Ni ratio, is 2/1 to 50/1, and preferably 2/1 to 20/1. The molar ratio of the Brønsted acid to the nickel compound is 0.25/1 to 10/1, and preferably 0.25/1 to 5/1.

According to a preferred method, the hydrocarbyl aluminum dihalide can be enriched with an aluminum trihalide, the mixture of the two compounds then corresponding to the formula AIRₙX₃₋ₙ, in which R is a hydrocarbyl radical comprising 1 to 12 carbon atoms, such as alkyl, aryl, aralkyl, alkaryl or cycloalkyl, X is a chlorine or bromine atom, and n is a number between 0 and 1. Suitable mixtures include: dichloroethyl aluminum enriched with aluminum chloride, the mixture having a formula AlEt_{0.9}Cl_{2.1}; dichloroisobutyl aluminum enriched with aluminum chloride, the mixture having a formula AliBu_{0.9}Cl_{2.1}; and dibromoethyl aluminum enriched with aluminum bromide, the mixture having a formula AlEt_{0.9}Br_{2.1}.

The reaction for oligomerization of ethylene can be implemented at a temperature of −20 to 80 °C , preferably 40 to 60 °C , under pressure conditions such that the reagents are kept at least for the most part in the liquid phase or in the condensed phase. The pressure is generally between 0.5 and 5 MPa, preferably between 0.5 MPa and 3.5 MPa. The time of contact is generally between 0.5 and 20 hours, preferably between 1 and 15 hours.

The oligomerization stage can be implemented in a reactor with one or more reaction stages in a series, with the ethylene feedstock and/or the catalytic composition that is preferably pre-conditioned in advance being introduced continuously, either in the first stage, or in the first stage and any other one of the stages. At the outlet of the reactor, the catalyst can be deactivated, for example by injection of ammonia and/or an aqueous solution of soda and/or an aqueous solution of sulfuric acid. The unconverted olefins and alkanes that are optionally present in the feedstock are then separated from the oligomers by a separation stage, for example by distillation or washing cycles by means of caustic soda and/or water.

The conversion per pass is generally 85 to 98%. The selectivity of n-butenes that are formed is generally between 50 and 80%. The n-butenes consist of butene-2 (cis-and trans-) and butene-1.

The effluent generally contains less than 0.2% by weight of isobutene, or even less than 0.1% by weight of isobutene.

### Separation of a Stream Rich in n-Butenes

The effluent that is obtained by dimerization of ethylene is subjected to a separation stage in such a way as to obtain an n-butene-enriched fraction.

The separation can be carried out by evaporation, distillation, extractive distillation, extraction by solvent or else by a combination of these techniques. These processes are known by one skilled in the art. Preferably, a separation of the effluent that is obtained by oligomerization of ethylene is carried out by distillation.

Preferably, the effluent of the oligomerization is sent into a distillation column system comprising one or more columns that makes it possible to separate, on the one hand, n-butenes from ethylene, which can be returned to the oligomerization reactor, and heavier olefins with 5 carbon atoms and more.

The higher olefins may subjected to hydrogenation so as to obtain renewably-sourced naphtha. “Renewably-sourced naphtha” shall mean naphtha produced from renewable sources. It is a hydrocarbon composition, consisting of mainly paraffins. The molecular weight of this renewably-sourced naphtha may range from hydrocarbons having 5 to 8 carbon atoms. Renewably-sourced naphtha can be used as a feedstock in steam-cracking to produce renewably-sourced light olefins, dienes and aromatics.

Hence, in an embodiment, step b)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream;
fractionating the effluent to recover a stream consisting essentially of n-butenes, a stream consisting essentially of heavier olefins, and an optional ethylene stream; and
subjecting the stream consisting essentially of heavier olefins to hydrogenation so as to obtain renewably-sourced naphtha.

### Metathesis of Ethylene With N-butenes

Ethylene is able to undergo metathesis with n-butenes to produce propylene. In one aspect of the invention, step b)-(ii) comprises a metathesis reaction between n-butenes obtained according to step (i) and ethylene to obtain propylene. The n-butenes obtained according to ethylene dimerization (i) are a mixed stream including 1-butene and 2-butenes. Essentially only the 2-butenes react in a metathesis reaction, while 1-butene is essentially inert.

In one embodiment, 1-butene is removed from the mixed stream of 1-butene and 2-butenes and directed to a use elsewhere in the plant. Thus, in one embodiment, step b)-(ii) comprises removal of 1-butene from the mixed stream to obtain a stream rich in 2-butenes, and subjecting the stream rich in 2-butenes to the metathesis reaction. A stream rich in 2-butenes may comprise at least 90 wt.-% of 2-butenes, based on the total amount of n-butenes.

Alternatively, 1-butene may be converted to 2-butene by double bond isomerization. Double bond isomerization is an equilibrium-limited reaction. It is thus advantageous to subject the mixed stream of n-butenes to metathesis so as to react 2-butene with ethylene prior to double bond isomerization of 1-butene. Hence, in one embodiment the n-butenes are a mixed stream including 1-butene and 2-butenes, and b)-(ii) comprises
b)-(iia) subjecting the mixed stream to the metathesis reaction to obtain propylene and unreacted 1-butene;
b)-(iib) subjecting the unreacted 1-butene to double bond isomerization to obtain 2-butenes; and
b)-(iic)recycling the 2-butenes obtained in step b)-(iib) to step b)-(iia).

In another embodiment, it is possible to convert 1-butene to 2-butene simultaneously with the metathesis reaction. For this purpose, a metathesis catalyst and an isomerization catalyst may be physically mixed or provided as distinct layers to allow both reactions to proceed simultaneously. Thus, in one embodiment, the n-butenes are a mixed stream including 1-butene and 2-butenes, and step b)-(ii) comprises passing the mixed stream through a metathesis/isomerization zone comprising both a metathesis catalyst and an isomerization catalyst. As 2-butene is consumed due to the metathesis reaction over the metathesis catalyst, it is thus replenished by isomerization of 1-butene to 2-butene over the isomerization catalyst.

The reaction is carried out in the presence of a metathesis catalyst on the basis of a metal which is selected from tungsten, molybdenum, rhenium, niobium, tantalum, vanadium, ruthenium, rhodium, iridium, osmium and nickel and the like. Tungsten, molybdenum and rhenium are preferred and tungsten is particularly preferred. Typically, tungsten catalysts are supported on silica, molybdenum and rhenium are supported on alumina based carriers. Especially preferred metathesis catalysts are WO₃-based catalysts, for example silica-supported WO₃ in the form of granules.

Suitable isomerization catalysts include magnesium-based catalysts such as MgO-based catalysts, for example tableted MgO.

Metathesis is carried out under conditions effective to produce an effluent comprising propylene, unconverted ethylene, and optionally 1-butene.

Unconverted ethylene and/or unconverted n-butenes may be recycled and combined with fresh ethylene and n-butenes to provided the metathesis feedstock.

The reaction may be conducted at 340-375 °C, 25-40 bar, a weight hourly space velocity (WHSV) of 7.5-30 hr⁻¹, and an ethylene to 2-butene molar ratio of 3:1 to 10:1. The reactor effluent may be sent to a deethenizer to remove C2 and lighter material. The bottoms from the deethenizer are sent to the depropenizer. High-purity, polymer-grade propylene (>99.9% molar purity) is recovered from the depropenizer overhead. The lighter material from the deethenizer and heavier C₄₊ material from the depropenizer are partly recycled to the reactors. Purge streams are provided for the lighter and heavier material to prevent buildup of inerts.

It should be noted that propane is not produced during the metathesis reaction. Consequently, polymer-grade propylene can be produced from the process, without the need for an expensive propylene-propane superfractionator.

Commercial processes for producing polymer-grade propylene by metathesis from ethylene and butenes feedstock are available from CB&I/Lummus (tradnemame OCT™) and from LyondellBasell.

### Skeletal Isomerization of n-Butenes

In one aspect, the process of the invention involves isomerization of n-butenes obtained according to (i) to obtain isobutene in accordance with step b)-(iii).

As the isomerization is an equilibrium reaction, the reaction mixture invariably contains unreacted n-butenes.

Expediently, step b)-(iii) comprises:
contacting the n-butenes with a skeletal isomerization catalyst in an isomerization zone to produce a mixture of n-butenes and isobutene;
recovering from the mixture a stream consisting essentially of n-butenes and a stream consisting essentially of isobutene; and
recycling the stream consisting essentially of n-butenes into the isomerization zone.

A suitable recovery scheme utilizes the reaction of isobutene with alkanol to produce alkyl tertiary butyl ether. The etherification reaction is selective with respect to isobutene, while n-butenes are unreactive in the reaction. The reaction therefore can be utilized as a method to separate n-butenes and isobutene.

Hence, isobutene may be recovered from the mixture of n-butenes and isobutene by the following steps:
(a) reacting the mixture of n-butenes and isobutene with isobutanol in the presence of an acidic ion exchange resin in an etherification unit to form a mixture of isobutyl tert-butyl ether (IBTBE) and unconverted n-butenes;
(b) distilling the reaction mixture in a first distillation unit to obtain a top product stream consisting essentially of n-butenes, and a bottom product comprising IBTBE;
(c) feeding the bottom product to a ether cleavage unit to decompose the IBTBE to obtain isobutene and isobutanol;
(d) distilling the mixture of isobutene and isobutanol produced in step (c) in a second distillation unit to obtain a top product stream consisting essentially of isobutene, and a bottom product comprising isobutanol; and
(e) Recycling the Bottom Product of Step (d) to Step (a).

Skeletal isomerization generally requires acidic catalysts. Known skeletal isomerization catalysts include aluminas and halogenated aluminas, particularly For Cl-promoted aluminas.

Certain zeolites have been shown to be highly effective in skeletal isomerization of normal olefins. Such zeolites include those selected from the group consisting of zeolites having the framework structure of ZSM-22, ZSM-23, and ZSM-35.

Examples for high selectivity, high stability catalysts are chlorinated γ-Al₂O₃, ferrierite SAPO-11 (silico-alumino phosphate molecular sieve) and MeAPO-11 (Me=Co, Mn, Mg) (molecular sieve). A particularly preferred catalyst is ferrierite. The typical elemental composition of ferrierite zeolite is Na₂Mg₂[Al₆Si₃₀O₇₂]·18H₂O as, for example, disclosed in U.S. Pat. No. 6,323,384.

Spent catalysts can be regenerated by heating in an oxygen-containing gas, such as air, at temperatures ranging from about 200 °C to about 700 °C.

Skeletal isomerization of n-butenes to isobutene is an equilibrium controlled process where equilibrium conversion decreases with increasing temperature.

The skeletal isomerization is carried out by contacting the feed with the catalyst, using any suitable contacting techniques, at temperatures at which skeletal isomerization of the feed of n-butenes occurs. The feed is preferably maintained in the vapor phase during contacting. The reactor temperature is preferably in the range of about 300° to about 650 °C, more preferably about 400° to about 580 °C. The weight hourly space velocity (WHSV) is not narrowly critical but will generally be within the range of about 0.1 to about 40 hr⁻¹, preferably from about 1 to about 20 hr⁻¹. Any convenient pressure can be used, with the lowest practical pressure preferred in order to minimize side reactions such as polymerization. Preferred pressures are within the range of about 0.1 to about 10 atmospheres, more preferably about 1 to about 4 atmospheres.

The equilibrium may not be achieved in the case of a single contact of the feed with the catalyst. However, in a particular variant of the process, the product stream leaving the catalyst bed can be divided up, and only one part is directly conveyed to the working-up process, while the other part is again conducted over the catalyst bed.

Several commercial processes for n-butene isomerization are known. In one embodiment the n-butene feedstock is vaporized, in general by heat exchange with reactor effluent, and further heated to reaction temperature. In the reactor, vapor reacts with up to 44% of n-butenes converted to isobutylene with greater than 86% selectivity. Typically, two reactors are cyclically operated: one in reaction mode, the other in regeneration mode. Reactor effluent is cooled, compressed and fractionated. Heavy ends are separated and removed as bottoms from the overhead isobutylene product.

Operating conditions, process and catalyst modifications are, for example, disclosed in U.S. Pat. Nos. 6,111,160 and 6,323,384. Typical operating conditions are: 340-360 °C reaction temperature, WHSV of 2 H⁻¹, an olefin partial pressure of 1-2 bar, and a total pressure of 1-3 bar.

### Hydroformylation of Propylene

In an aspect of the invention, the renewably-sourced C₃₋₄-olefin is propylene, wherein step c) comprises hydroformylation of the propylene to produce n-butyraldehyde, isobutyraldehyde or a mixture thereof.

The hydroformylation of propylene may be carried out according to any known process. If desired, the produced aldehydes can be separated by fractionation.

Hydroformylation or the oxo process is an important large-scale industrial process for preparing aldehydes from olefins, carbon monoxide and hydrogen. These aldehydes can optionally be hydrogenated with hydrogen in the same operation or subsequently in a separate hydrogenation step, to produce the corresponding alcohols. In general, hydroformylation is carried out in the presence of catalysts which are homogeneously dissolved in the reaction medium. Catalysts used are generally the carbonyl complexes of metals of transition group VIII, in particular Co, Rh, Ir, Pd, Pt or Ru, which may be unmodified or modified with, for example, amine-containing or phosphine-containing ligands. A summarizing account of the processes practiced on a large scale in industry is found in J. Falbe, “New Syntheses with Carbon Monoxide”, Springer Verlag 1980, p. 162 ff., U.S. Pat. No. 3,527,809; 3,917,661; 4,148,830; 4,742,178, 4,769,984; 4,885,401; 6,049,011.

Propylene is preferably hydroformylated using ligand-modified rhodium carbonyls as the catalyst. Hydroformylation of propylene can be carried out at temperatures in the range of 50 °C to 200 °C , preferably 60 °C to 150 °C , and more preferably 70 °C to 120 °C.

In one embodiment, the hydroformylation reaction is conducted at a low pressure, e.g., a pressure in the range of 0.05 to 50 MPa (absolute), and preferably in the range of about 0.1 MPa to 30 MPa, most preferably at a pressure below 5 MPa. Desirably, the partial pressure of carbon monoxide is not greater than 50% of the total pressure.

The proportions of carbon monoxide, hydrogen, and ethylene in the hydroformylation reaction medium can be selected within a wide range. In some embodiments, based on the total amount of CO, hydrogen, and propylene, CO is from about 1 to 50 mol-%, preferably about 1 to 35 mol-%; H₂ is from about 1 to 98 mol-%, preferably about 10 to 90 mol-%; and ethylene is from about 0.1 to 35 mol-%, preferably about 1 to 35 mol-%.

The hydroformylation reaction preferably takes place in the presence of both liquid and gas phases. The reactants generally are in the gas phase. The catalyst typically is in the liquid phase. Because the reactants are gaseous compounds, a high contact surface area between the gas and liquid phases is desirable to enhance good mass transfer. A high contact surface area between the catalyst solution and the gas phase may be provided in any suitable manner. In a batch process, the batch contents are thoroughly mixed during the course of the reaction. In a continuous operation the reactor feed gas can be contacted with the catalyst solution in, for example, a continuous-flow stirred autoclave where the gas is introduced and dispersed at the bottom of the vessel, preferably through a perforated inlet (e.g., a sparger). High contact between the catalyst and the gas feed may also be provided by dispersing the solution of the Rh catalyst on a high surface area support, a technique well known in the art as supported liquid phase catalysis, or providing the Rh as part of a permeable gel.

The reaction may be conducted either in a batch mode or, preferably, on a continuous basis. One or more reactors may be used in continuous modes to carry out the reaction in one or more stages.

The ratio of H₂ to CO in the syngas used for hydroformylation is desirably in the range from 1.1:1 to 1.01:1, preferably 1.06:1 to 1.02:1. Often, syngas may be made or otherwise initially provided in a manner such that the ratio of hydrogen to CO is much higher than this. The excess hydrogen can be separated and used in other reaction stages as desired.

The hydroformylation process inherently produces high boiling liquid aldehyde condensation by-products, e.g. dimers, trimers and tetramers, which may serve as a solvent for the hydroformylation process, as well as other liquid heavies. Thus a small amount of such higher boilers is always invariably contained in the crude aldehyde product mixture obtained even after separating the initial aldehyde product from its lights, e.g. carbon monoxide, hydrogen, unreacted alkylene, alkane by-product, etc., as in the case of a continuous gas recycle hydroformylation process or after separating the initial aldehyde product from its lights and catalyst containing solution as in the case of a continuous liquid recycle hydroformylation process. Indeed even after separating the lower boiling, branched chain aldehyde from its higher boiling normal straight chain aldehyde counterpart in order to obtain purified branched chain aldehyde (e.g. iso-butyraldehyde) and leave the straight chain aldehyde (e.g. n-butyraldehyde), the normal aldehyde product may still contain a higher amount of such organic heavies than desired for its eventual end-use.

The conventional procedure to separate the branched-chain aldehyde product from the straight chain aldehyde product of such crude aldehyde product mixtures resulting from conventional continuous rhodium catalyzed hydroformylation processes is by a two step distillation procedure that involves the use of two separate distillation columns. For example, purified branched chain aldehyde (iso-butyraldehyde) is first separated from the crude aldehyde product mixture via distillation in an initial distillation column and then the remaining normal (straight chain) aldehyde (n-butyraldehyde) is further refined or purified from any remaining higher boiling by-products by a second distillation carried out in a second distillation column.

Alternatively, the distillation regime is modified to recover a portion of the straight chain aldehyde in the first distillation column. Hence, the crude aldehyde product mixture starting material may be fed to a distillation column and distilled in said distillation column, so as to concurrently obtain (i) a liquid aldehyde product stream taken from at or near the top of the distillation column and consisting essentially of purified branched chain aldehyde and (ii) a volatilized aldehyde product stream consisting essentially of purified straight chain aldehyde as a vapor sidestream in an amount of no more than about 70 percent by weight of the amount of straight chain aldehyde present in said liquid crude aldehyde product mixture starting material, and (iii) wherein the remaining liquid aldehyde consisting essentially of straight chain aldehyde is recovered from at or near the bottom of the distillation column together with the organic heavies present in the liquid crude aldehyde product mixture starting material. The remaining liquid aldehyde is directed to a butyraldehyde residue tower, where straight chain aldehyde is withdrawn overhead and remaining heavies are recovered from at or near the bottom of the butyraldehyde residue tower.

The organic heavies recovered from the crude aldehyde product mixtures include any organic solvent and organic by-product having boiling points above that of the straight chain aldehyde product compounds, such as the liquid aldehyde condensation by-products (dimers, trimers, tetramers, etc.), and other common higher boiler by-product, e.g. corresponding alkanol. Of course it is understood that such crude aldehyde product mixtures can also contain some minor amounts of residual lights (e.g. unreacted olefin and by-product alkane) and organophosphorus contaminant e.g. free organophosphorus ligand and/or its corresponding oxide.

According to the invention, the organic heavies recovered from the crude aldehyde product mixtures constitute a waste stream that is incinerated to generate thermal energy.

### Reaction of Isobutene with Formaldehyde to Produce Isoprenol

In another aspect of the invention, the renewably-sourced C₃₋₄-olefin is isobutene, and step c) comprises a reaction of isobutene with formaldehyde to produce isoprenol.

The production of isoprenol (3-methyl-3-butene-1-ol) is well-known. For example, it can be produced by a Prins reaction between isobutene and formaldehyde in liquid phase at temperatures of 220 to 280 °C and a pressure of 230 to 270 bar with or without a catalyst. The reaction mixture may be fractionated to obtain isoprenol. Further details are provided in WO 2008/037693.

Purification of the Crude Isoprenol Stream May Comprise
(i) directing the stream of crude isoprenol to a first low-boiler separation tower operated at a pressure of 1.5 bara or lower, to obtain a first bottoms stream containing isoprenol and formaldehyde, and a first distillate stream containing water and low-boilers;
(ii) directing the first bottoms stream to a second low-boiler separation tower operated at a pressure of 2 bara or higher, to obtain a second distillate stream containing aqueous formaldehyde, and a second bottoms stream containing isoprenol; and
(iii) directing the second bottoms stream to a finishing tower to obtain pure isoprenol as a distillate stream, and a bottoms stream containing high-boilers.

### Isomerization of Isoprenol to Prenol

In one embodiment of this aspect, step c) further comprises an isomerization of isoprenol to prenol.

The isomerization is carried out in the presence of hydrogen and a catalyst. A preferred catalyst is a fixed bed catalyst containing palladium and selenium or tellurium or a mixture of selenium and tellurium supported on silicium dioxide. The isomerization is carried out at a temperature of 50 to 150 °C to produce a reaction mixture of prenol and isoprenol. The isoprenol can be recycled. Further details are provided in WO2008037693.

### Oxidation of Isoprenol to Isoprenal

In another embodiment of this aspect, step c) further comprises an oxidation reaction of the isoprenol to produce isoprenal.

The selective oxidation of primary alcohols such as isoprenol to the corresponding aldehyde is a well-known reaction. Isoprenol can be oxidized to isoprenal by oxidative dehydrogenation by means of an oxygen-containing gas under catalysis, for example a supported copper, silver and/or gold catalyst, preferably a silver catalyst. The oxidation is carried out at a reaction temperature of 300 to 500 °C and results in a mixture of isoprenal (3-methyl-3-butenal) and prenal (3-methyl-2-butenal) and unreacted isoprenol. The mixture contains an excess of isoprenal, for example in a wt-ratio of 2:1 to 5:1. Further details are provided in WO2008037693.

### Isomerization of Isoprenal to Prenal

In another embodiment of this aspect, step c) further comprises isomerization of isoprenal to prenal.

Isoprenal is subjected to isomerization in order to produce additional prenal, wherein the isoprenal is preferably used in the form of the mixture obtained from step (b). The isomerization is carried out in the presence of an isomerization catalyst, preferably sodium acetate, at a temperature of 100 to 200 °C to produce a mixture of prenal and isoprenol. The mixture is fractionated to produce a prenal stream and an isoprenol stream. The isoprenol can be recycled. Further details are provided in WO2008037693.

### Reaction of Prenol With Prenal to Produce Citral

In another embodiment of this aspect, step c) further comprises conversion of the isoprenol the isoprenal to produce citral.

Prenol and prenal are first subjected to an acetalization to produce the diprenol acetal of prenal, 3-methyl-2-butenal-diprenylacetal. The acetalization is carried out under vacuum and acidic catalysis, for example a mineral acid, such as nitric acid or sulfuric acid, at a temperature up to 100-120 °C. The water formed during acetalization is continuously removed.

The acetal is then subjected to thermal cleavage in the presence of an acidic catalyst such as phosphoric acid, at a temperature of 150-170 °C to obtain cis/trans-prenyl-(3-methyl-butadienyl) ether. Under the reaction conditions this ether undergoes a Claisen and Cope rearrangement to give citral. Further details are provided in WO2008037693.

Suitably, the cleaving may be carried out in the lower part or the sump of the distillation column acting as cleaving column. Preferably, the acetal is introduced into the lower part of the distillation column, into the sump of the distillation column or into the evaporator of the distillation column. Typically, the bottoms from the cleaving column are a mixture of high boilers which are comprised of C5-oligomers resulting from the thermal instability of the diprenyl acetal. Part of the bottoms from the cleaving column is continuously withdrawn. This serves to avoid accumulation of high boilers.

The invention is further illustrated by the appended drawing and the example that follows.

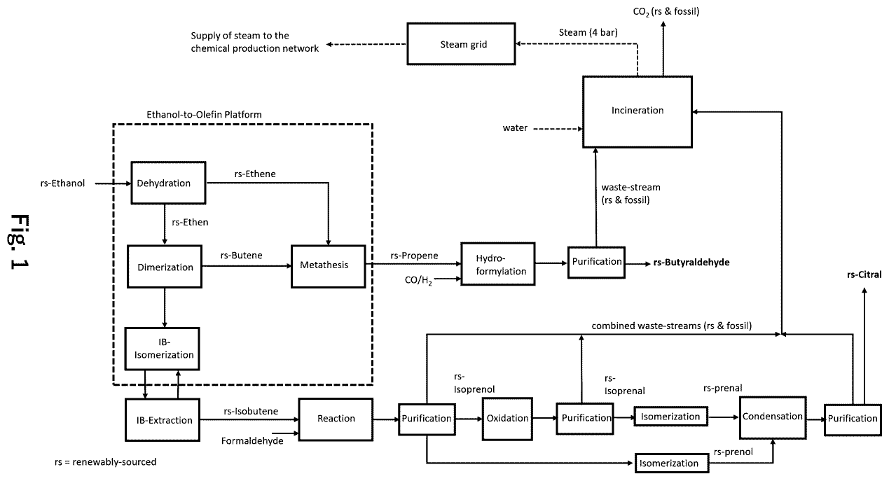

FIG. 1 is a schematic diagram of a production plant for the integrated production of butyraldehyde and citral starting from renewably sourced ethanol.

As shown in FIG. 1, an integrated production facility includes an ethanol-to-olefin platform having an ethanol dehydration unit, an ethylene dimerization unit, a metathesis unit, and a butene isomerization unit. The facility includes an isobutene extraction unit which recovers from the mixed stream of n-butenes and isobutene received from the butene isomerization unit a stream consisting essentially of n-butenes and a stream consisting essentially of isobutene, wherein the stream consisting essentially of n-butenes is recycled to the butene isomerization unit.

Propylene formed in the metathesis unit is subjected to hydroformylation with syngas (CO/H₂) in a hydroformylation unit. The obtained crude hydroformylation product is subjected to purification in a butyraldehyde purification unit producing pure butyraldehyde and a waste stream. The waste stream is directed to an incineration unit.

Isobutene withdrawn from the isobutene extraction unit is reacted with formaldehyde in a reaction unit to produce a crude isoprenol stream, the obtained crude isoprenol stream is subjected to purification in a purification unit to produce a purified isoprenol stream and a first waste stream. The purified isoprenal stream is subjected to an oxidation reaction in an oxidation unit to produce a crude isoprenal stream, the obtained crude isoprenal stream is subjected to purification in a purification unit to produce a purified isoprenal stream and a second waste stream. After isomerization of the purified isoprenal stream to produce prenal, the prenal is reacted with prenol in a condensation unit to produce a crude citral stream. Prenol is obtained by isomerization of purified isoprenol. The obtained crude citral stream is subjected to purification in a purification unit to produce a purified citral stream and a third waste stream. The first waste stream, second waste stream and third waste stream are combined and directed to the incineration unit.

In the incineration unit, the thermal energy produced by the incineration of the waste streams is used to vaporize and heat water to produce heated steam which is fed to a steam grid.

### Example (prophethical)

In a facility according to FIG. 1, one ton of each butyraldehyde and citral are produced. 3.7 tons of renewably sourced ethanol are consumed for this purpose.

0.6 tons of propylene formed in the metathesis unit is subjected to hydroformylation with 0.4 tons of syngas (fossil origin) in the hydroformylation unit. Purification of the obtained crude hydroformylation product yields a total waste stream of 0.02 tons of mixed fossil/renewable origin, 0.015 tons being of renewable origin.

1.1 tons of isobutene withdrawn from the isobutene extraction unit are reacted with 0.6 tons of formaldehyde (fossil origin) in the reaction unit and the obtained isoprenol is further processed to produce citral, including purification of intermediates in the purification units yielding a first waste stream, second waste stream and third waste stream totaling to a combined stream of 0.4 tons of mixed fossil/renewable origin, 0.3 tons being of renewable origin.

Incineration of the combined waste streams from the butyraldehyde purification and citral production produces 0.9 tons of carbon dioxide of mixed fossil/renewable origin, 0.7 tons being of renewable origin, amounting to a reduction of about 70% of carbon dioxide of fossil origin. In the incineration unit, 5.4 tons of heated steam (4 bar) are produced, being the heat equivalent of the incineration of 0.45 tons of ethanol.

1.-16. (canceled)

17. A process for the manufacture of a chemical of interest, said process comprising the steps of:
a) subjecting a feedstock comprising a renewably-sourced ethanol to dehydration to produce a renewably-sourced ethylene stream;
b) subjecting the renewably-sourced ethylene stream to an olefin-interconversion, to obtain one or more renewably-sourced C₃₋₄-olefins, selected from propylene, n-butenes and isobutene; the olefin-interconversion comprising (i) and, where required, one or both of (ii) and (iii):
(i) ethylene dimerization to obtain n-butenes;
(ii) metathesis reaction between n-butenes obtained according to (i) and ethylene to obtain propylene; and
(iii) isomerization of n-butenes obtained according to (i) to obtain isobutene; and
c) subjecting the renewably-sourced C₃₋₄-olefin to a chemical conversion or sequence of chemical conversions to obtain the chemical of interest, the chemical conversion or sequence of chemical conversions producing one or more crude product streams and, optionally, one or more crude intermediate product streams,
d) subjecting at least one of the crude product stream(s) and optional intermediate crude product stream(s) to purification producing one or more purified product streams, one or more waste streams and, optionally, one or more purified intermediate product streams,
e) incinerating at least one of the waste streams to produce thermal energy.

18. The process according to claim 17, comprising f) transferring at least part of the thermal energy to a water stream to generate a heated steam stream.

19. The process according to claim 18, wherein the heated steam stream is used to provide heat to a heat-accepting process.

20. The process according claim 19, wherein the heated steam stream is used to provide at least a portion of the heat consumed in at least one of steps a) and b).

21. The process according to claim 17, wherein the waste that is incinerated according to step e) has a lower heating value in the range of from 10 to 45 MJ per kg of waste.

22. The process according to claim 17, wherein the percentage of biogenic carbon in the waste stream that is incinerated according to step e) is in the range from 20 to 100 % based on the total carbon in the waste.

23. The process according to claim 17, wherein purification involves distillation of the crude product stream(s) or crude intermediate product stream(s).

24. The process according to claim 23, wherein at least one waste stream directed to incineration is a distillation bottoms stream.

25. The process according to claim 17, wherein step b)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream;
fractioning the effluent to recover a stream consisting essentially of n-butenes, a stream consisting essentially of heavier olefins, and an optional ethylene stream; and
subjecting the stream consisting essentially of heavier olefins to hydrogenation so as to obtain renewably-sourced naphtha.

26. The process according to claim 17, wherein the n-butenes are a mixed stream including 1-butene and 2-butenes, and wherein b)-(ii) comprises removal of 1-butene from the mixed stream to obtain a stream rich in 2-butenes, and subjecting the stream rich in 2-butenes to the metathesis reaction.

27. The process according to claim 17, wherein the n-butenes are a mixed stream including 1-butene and 2-butenes, and wherein b)-(ii) comprises
b)-(iia) subjecting the mixed stream to the metathesis reaction to obtain propylene and unreacted 1-butene;
b)-(iib) subjecting the unreacted 1-butene to double bond isomerization to obtain 2-butenes; and
b)-(iic) recycling the 2-butenes obtained in step b)-(iib) to step b)-(iia).

28. The process according to claim 17, wherein the n-butenes are a mixed stream including 1-butene and 2-butenes, and wherein b)-(ii) comprises passing the mixed stream through a metathesis/isomerization zone comprising both a metathesis catalyst and an isomerization catalyst.

29. The process according to claim 17, wherein step b)-(iii) comprises:
subjecting the n-butenes to skeletal isomerization to produce a mixture of n-butenes and isobutene;
recovering from the mixture a stream consisting essentially of n-butenes and a stream consisting essentially of isobutene; and
recycling the stream consisting essentially of n-butenes into the skeletal isomerization.

30. The process according to according to claim 17, wherein the renewably-sourced C₃₋₄-olefin is propylene, wherein step c) comprises hydroformylation of the propylene to produce a crude aldehyde stream comprising n-butyraldehyde and isobutyraldehyde; and step d) comprises purification of the crude aldehyde stream to produce at least one purified aldehyde stream and a waste stream.

31. The process according to according to claim 17, wherein the renewably-sourced C₃₋₄-olefin is isobutene, and step c) comprises (c-i) through (c-v) and step d comprises (d-i) through (d-iii):
(c-i) reacting isobutene with formaldehyde to produce a crude isoprenol stream, (d-i) purifying the crude isoprenol stream to produce a purified isoprenol stream and a first waste stream;
(c-ii) oxidizing the purified isoprenal stream to produce a crude isoprenal stream, (d-ii) purifying the crude isoprenal stream to produce a purified isoprenal stream and a second waste stream;
(c-iii) isomerizing the purified isoprenol stream to produce prenol,
(c-iv) isomerizing the purified isoprenal stream to produce prenal,
(c-v) reacting the prenol with prenal to produce a crude citral stream, (d-iii) purifying the crude citral stream to produce a purified citral stream and a third waste stream.

32. The process according to claim 31, wherein the first waste stream, second waste stream and third waste stream are combined and directed to incineration.
